(19) United States
(12) Patent Application Publication
(10) Pub. No.: US 2026/0242770 A1
(43) Pub. Date: Aug. 20, 2026

(54) DEVICE AND METHOD FOR RAPID EXTRACTION OF NUCLEIC ACIDS FROM COMPLEX SAMPLES

(71) Applicant: VedaBio, Inc., San Diego, CA (US)

(72) Inventors: Donald Masquelier, San Diego, CA (US); Phillip Belgrader, San Diego, CA (US); Francesca Pearson, San Diego, CA (US)

(21) Appl. No.: 18/847,663

(22) PCT Filed: Mar. 28, 2024
(86) PCT No.: PCT/US2024/022090

§ 371 Date: Sep. 16, 2024

Related U.S. Application Data
(60) Provisional application No. 63/456,450, filed on Mar. 31, 2023.

(51) Int. Cl. C12N 15/10; H01F 7/02

(52) U.S. Cl. CPC C12N 15/1013; H01F 7/02

(57) ABSTRACT

Automated sample prep devices and methods for the use thereof is presented. The automated sample prep devices are unique in that they accomplish cell lysis, nucleic acid fragmentation, and, in some embodiments, nucleic acid purification via bead beating using two diametrically magnetized magnets. The two magnets are set apart so that the magnetic fields of the magnets interact both statically and dynamically. A sample prep chamber is positioned between the magnets in the middle of the interacting magnetic fields. The sample chamber comprises a sample, as well as lysis/binding beads and a number of metal spheres. The spheres trigger the interaction or movement between both the spheres themselves and the lysis/binding beads as the magnetic fields are altered.

### RELATED APPLICATIONS

This application is a 371 US National Phase filing of International Application PCT/US24/22090, filed 28 Mar. 2024; which claims priority to U.S. Ser. No. 63/456,450, filed 31 Mar. 2023, both of which are incorporated by reference in their entirety.

### FIELD OF THE INVENTION

The present disclosure relates to methods and devices for automated sample prep, including cell and/or virus lysis, nucleic acid fragmentation and nucleic acid purification.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Rapid and accurate identification of, e.g., infectious agents, microbe contamination, variant nucleic acid sequences that indicate the present of diseases such as cancer, or contamination by heterologous sources is important in order to select correct treatment; identify tainted food, pharmaceuticals, cosmetics and other commercial goods; and to monitor the environment including identification of biothreats. Improved technologies and automated systems—including automated sample prep devices—that allow very rapid and accurate detection of nucleic acids are therefore needed for timely diagnosis and treatment of disease, to identify toxins in consumables and the environment, as well as in other applications.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present automated sample prep device is unique in that it accomplishes cell lysis, nucleic acid fragmentation and, in some embodiments, nucleic acid purification via bead beating using two diametrically magnetized cylindrical magnets (“magnets”). The two magnets are set apart so that the magnetic fields of the magnets interact both statically and dynamically. A sample prep chamber is positioned between the magnets between the interacting magnetic fields. The sample chamber comprises a sample, as well as lysis/binding beads (“lysis/binding beads”) and a number of metal (e.g., stainless steel) spheres (“spheres”). The spheres trigger interaction or movement between both the spheres and the lysis/binding beads as the magnetic fields are altered. The sample prep device thus is a modification of bead beaters devices known in the art.

Thus, there is provided in one embodiment a device for automated preparation of nucleic acids from a sample comprising: a first rotatable diametrically poled magnet; a second rotatable diametrically poled magnet, wherein the second magnet is set apart from the first rotatable magnet, and wherein one or both of the first and second magnets rotates around a vertical axis; at least one motor configured to rotate at least one of the first and second magnets; and a sample chamber positioned between the first and second rotatable magnet, wherein the sample chamber comprises lysis beads and at least X spheres, wherein the spheres have a magnetic permeability greater than 1.000, and wherein a diameter of the X spheres exceeds a diameter of the sample chamber.

In some aspects of this embodiment, the first and second rotatable magnets are cylindrical with a central hole vertically traversing the first and second rotatable magnets and in some aspects, the first and second rotatable magnets are fabricated from neodymium. In some aspects, the sample chamber has a volume of 0.2 mL to 10.0 mL, or 1.0 mL and 5.0 mL, or 1.5 mL and 2.5 mL.

In some aspects, the first and second magnets are configured to impart between a 1.5-pound magnetic pull force and a 10.0-pound pull force, or between a 2.0-pound magnetic pull force and a 7.0-pound magnetic pull force, or between a 3.0-pound magnetic pull force and a 5.0-pound magnetic pull force.

In some aspects of this embodiment, the spheres are ferrous metal spheres, and in some aspects, the spheres are stainless steel.

Another embodiment provides a method for isolating nucleic acids from cells or viral particles in a sample comprising the steps of: obtaining a sample; obtaining the sample prep device of claim 1; transferring the sample to the sample chamber of the sample prep device; adding nuclease inactivation buffer to the sample in the sample chamber; rotating at least one of the rotatable magnets at 500 and about 6000 RPM; diluting the sample with buffer; and collecting the sample. In some aspects of this embodiment, the at least one rotating magnet is rotated at about 1000 to 5000 RPM.

In some aspects of this embodiment, the nuclease inactivation buffer comprises EDTA, TCEP or DTT, and in some aspects, the rotating step is performed at 95° C.

In some aspects, a volume of the sample is 100 μL to 1 mL, and in some aspects a volume of the sample is 200-300 μL.

In some aspects, the lysis beads comprise 10-60% of the sample volume after the transferring step, and in some aspects, the lysis beads comprise 20-50% of the sample volume after the transferring step.

In some aspects, the lysing/binding buffer comprises a chaotropic agent, and in some instances, the chaotropic agent is guanidine thiocyanate.

In some aspects, after the adding step and before the rotating step, the method further comprises a step of heating the sample. In some aspects, after the rotating step and before the diluting step, the method further comprises a step of washing the lysis beads. In some aspects, after the washing step and before the diluting step, the method further comprises a step of suspending the lysis beads in suspension buffer and heating the suspension buffer to 95° C., and in some aspects, the suspension buffer comprises Tris at pH 10. In some aspects, after the suspending step and before the diluting step, the method further comprises a step of rotating at least one of the rotatable magnets at 500-6000 RPM for 0.5-5 minutes.

These aspects and other features and advantages of the invention are described below in more detail.

### BRIEF DESCRIPTION OF THE DRAWINGS

The foregoing and other features and advantages of the present invention will be more fully understood from the following detailed description of exemplary embodiments taken in conjunction with the accompanying drawings in which:

FIG. 1A is a simplified depiction of the attraction and opposition modes of the magnets employed in the sample prep device.

FIG. 1B is a perspective view of one embodiment of the sample prep device described herein.

FIG. 1C is a perspective partially transparent view of the embodiment of the sample prep device shown in FIG. 1B.

FIG. 1D is a cross-sectional or cut-away view of the embodiment of the sample prep device shown in FIGS. 1B and 1C.

FIG. 1E is a perspective partially transparent view of a second embodiment of the sample prep device described herein.

FIG. 1F is a cut-away perspective view of the second embodiment of the sample prep device shown in FIG. 1E.

FIG. 1G is a front perspective view of a third embodiment of a sample prep device.

FIG. 1H is a front perspective view of a fourth embodiment of a sample prep device.

FIG. 1I is a front perspective view of a fifth embodiment of a sample prep device.

FIG. 1J is an exemplary system diagram for the sample prep device.

FIG. 2 is a graphic depiction of two exemplary workflows for sample preparation in the automated sample prep device (“sample prep device”) according to the present disclosure.

FIG. 3 is a graphic depiction of an exemplary workflow and amplification plot for sample preparation in the sample prep device according to the present disclosure.

FIG. 4 is a graphic depiction of an alternative exemplary workflow and results for sample preparation in the sample prep device according to the present disclosure.

FIG. 5 consists of two amplification plots showing the percent recovery of both DNA and RNA using the methods and sample prep device described herein.

FIG. 6 shows the data obtained for nucleic acid fragmentation using the sample prep device, heat treatment at 95° C., and combining the sample prep device and heat treatment at 95° C.

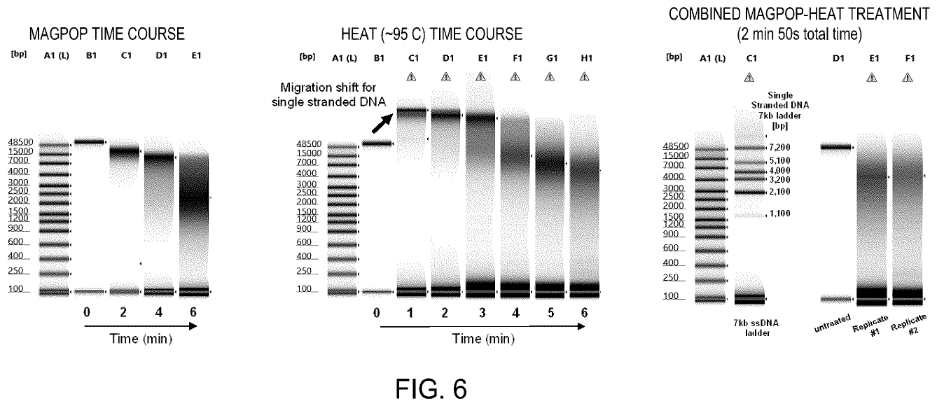

It should be understood that the drawings are not necessarily to scale, and that like reference numbers refer to like features.

### Definitions

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

All of the functionalities described in connection with one embodiment of the compositions and/or methods described herein are intended to be applicable to the additional embodiments of the compositions and/or methods except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Note that as used herein and in the appended claims, the singular forms “a,” “an,” and “the” include plural referents unless the context clearly dictates otherwise. Thus, for example, reference to “a cell” refers to one or more cells, and reference to “a system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention. Conventional methods are used for the procedures described herein, such as those provided in the art, and demonstrated in the Examples and various general references.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both limits, ranges excluding either or both of those included limits are also included in the invention.

The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context.

As used herein, the term “contacting” refers to placement of two moieties in direct physical association, including in solid or liquid form. Contacting can occur in vitro with isolated cells (for example in a tissue culture dish or other vessel) or in samples or in vivo by administering an agent to a subject.

A “control” is a reference standard of a known value or range of values.

As used herein, the term “sample” refers to cells or viruses contained in peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. “Sample” may also refer to specimens or aliquots from food; agricultural products; pharmaceuticals; cosmetics; nutraceuticals; personal care products; environmental substances such as soil, water (from both natural and treatment sites), air, or sewer samples; industrial sites and products; and chemicals and compounds. A sample further may include a homogenate, lysate or extract.

### DETAILED DESCRIPTION

The present disclosure provides an automated sample prep device and methods of using the sample prep device for sample preparation of cellular or viral nucleic acids in less than 10 minutes. The present automated sample prep device is unique in that it accomplishes cell lysis, nucleic acid fragmentation and, in some embodiments, nucleic acid purification via bead beating using two diametrically magnetized cylindrical magnets (“magnets”). The two magnets are set apart so that the magnetic fields of the magnets interact both statically and dynamically. A sample prep chamber is positioned between the magnets in the middle of the interacting magnetic fields.

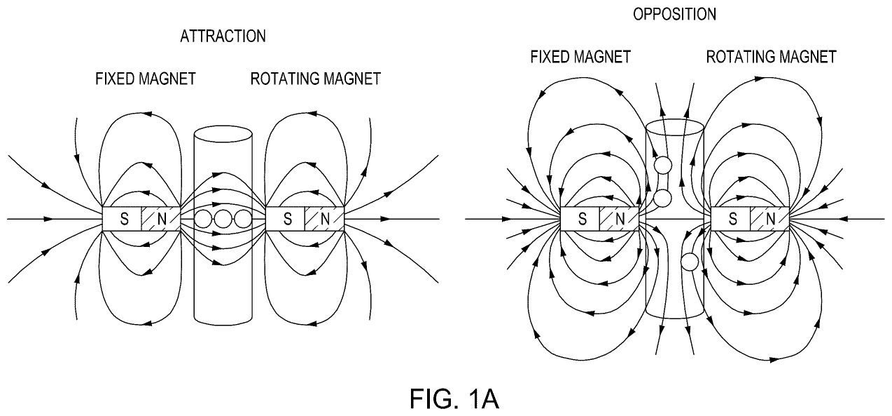

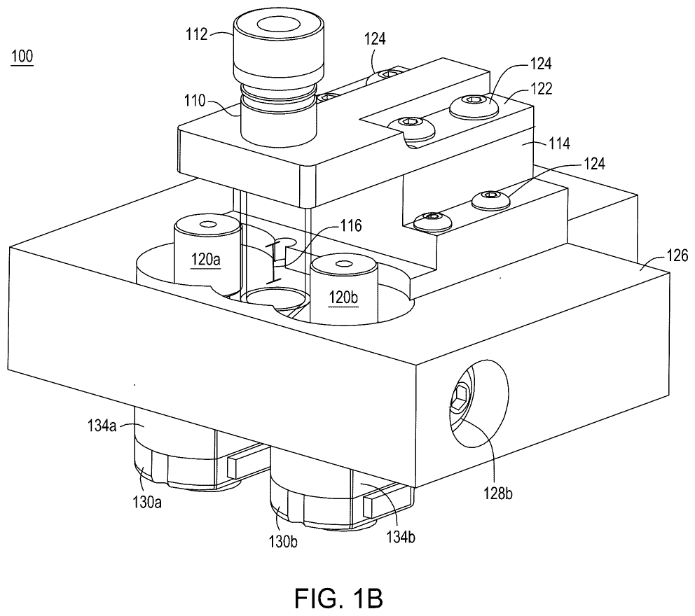

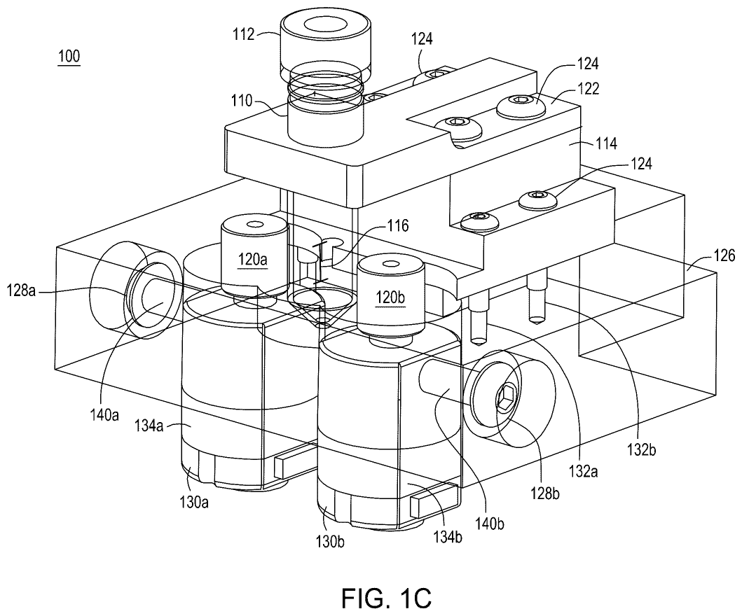

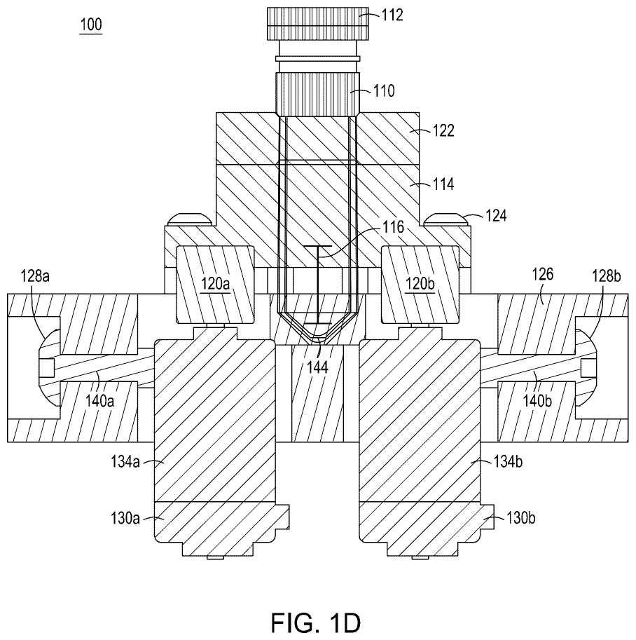

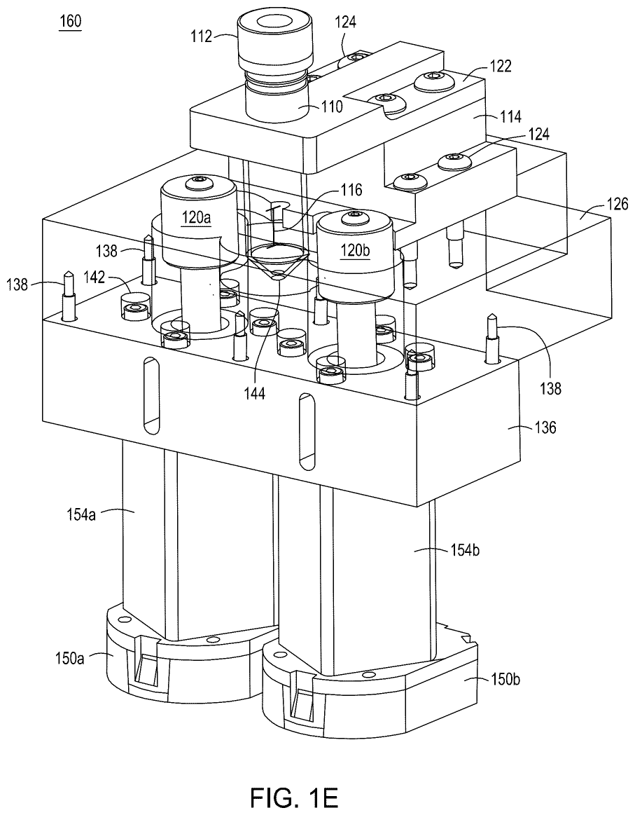

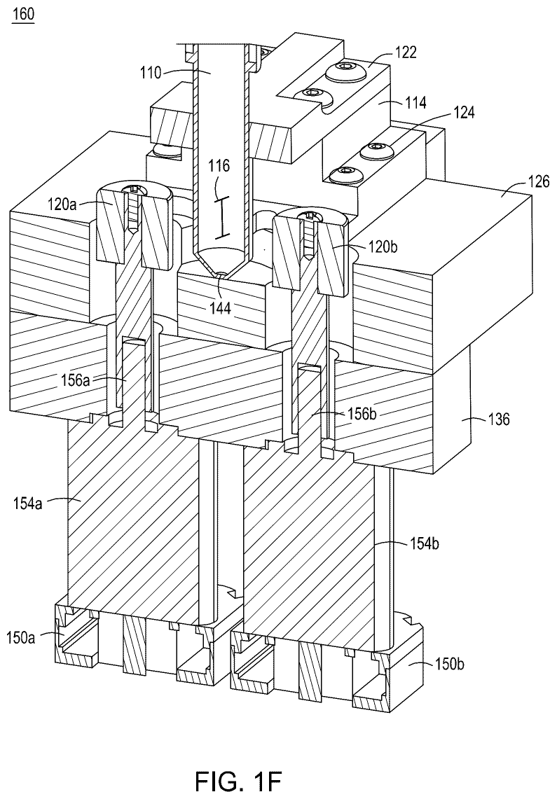

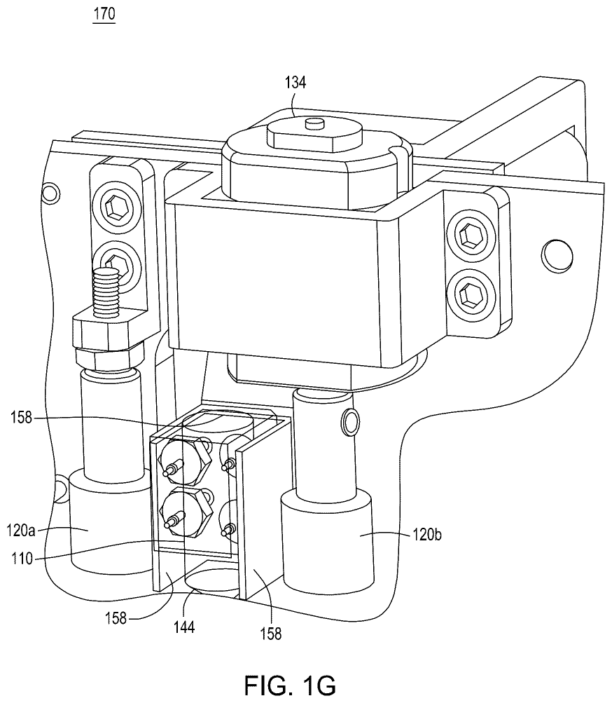

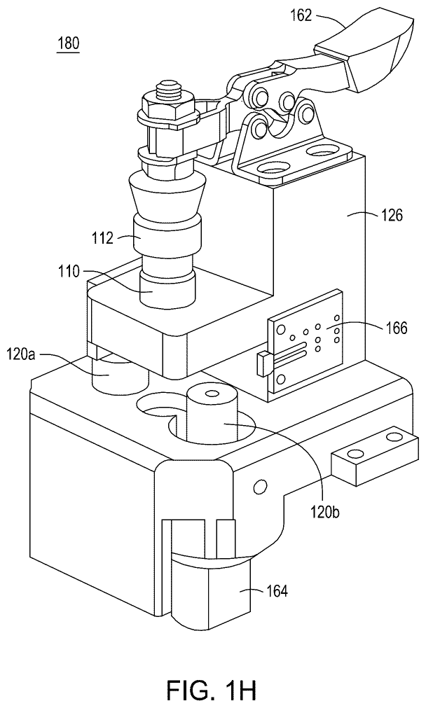

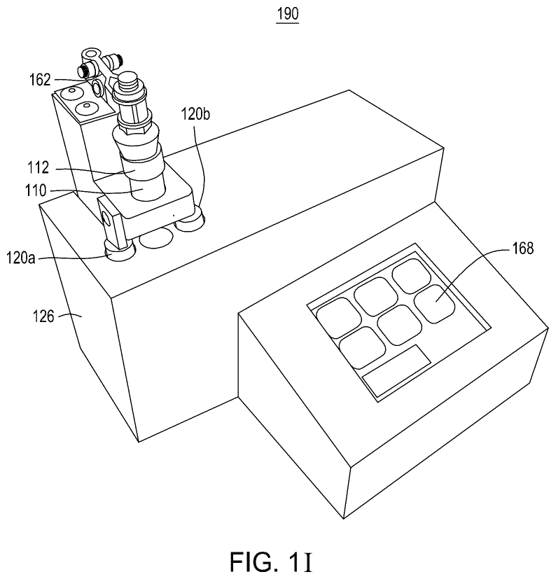

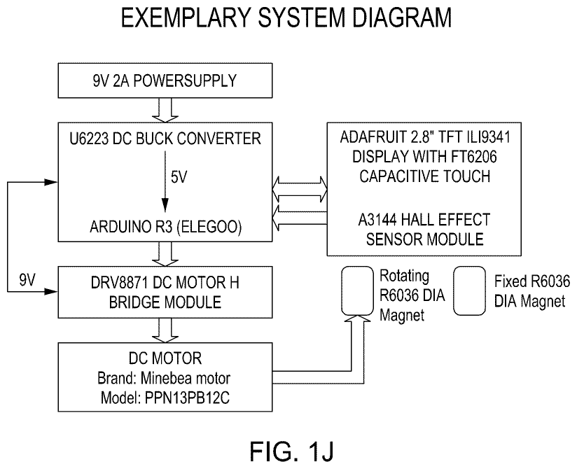

FIG. 1A is a simplified depiction of exemplary attraction and opposition modes of the magnets employed in the sample prep device. In this example, there is one fixed magnet and one rotating magnet. In FIG. 1A, positioned between the fixed and rotating magnets is a chamber comprising metal spheres. When the fixed and rotating magnets are in the “attraction” position (at left), the metal spheres align with the magnetic fields; however, the metal spheres then move to follow the field lines when the magnets are moved to the “opposition” position (at right). As the metal spheres move within the confines of the chamber, they collide with contents of the chamber.

In practice, the sample chamber comprises a sample, as well as lysis/binding beads (“lysis/binding beads”) and a number of metal (e.g., stainless steel) spheres with a low degree of magnetic response or permeability. The diameter of the metal spheres is selected such that X number of metal spheres will exceed the diameter of the sample chamber. Ensuring the span of the collective diameter of the metal spheres is greater than the span of the diameter of the sample chamber enhances the interaction or movement of the spheres as the magnetic fields are altered. One or both of the magnets are rotated on their cylindrical axis and the metal spheres change position rapidly as they move in and out of the changing magnetic field lines. The movement of the metal spheres then causes chaotic movement and collisions with and between the metal spheres themselves as well as the lysis/binding beads, thereby lysing cells and/or viral particles in the sample and fragmenting the nucleic acids released from the lysed cells and viruses. Thus, the sample prep device describe herein performs bead beating, albeit in a unique manner. Further, the sample prep device works on small samples in a sample chamber that need not be filled, unlike known commercial devices. Typically, at least one rotating magnet is rotated between 1000 and 10000 times per minute, or 2000 and 9000 times per minute, or 3000 and 7000 times per minute, or 3500 and 5000 times per minute. In some embodiments, both magnets may be rotated, where the magnets can be rotated at the same or at a different rate. Additionally, the fixed magnet can rotate to a new fixed position during a protocol to change the motion of the beads.

The nucleic acids may be DNA, RNA, or cDNA molecules or a mixture thereof. The nucleic acids originate from source organisms that are present in a sample, such as a biological sample from a subject (including non-human animals or plants), items of manufacture, or an environmental sample (e.g., water or soil). Non-limiting examples of biological samples include blood, serum, plasma, saliva, mucus, a nasal swab, a buccal swab, stool, a cell, a cell culture, and tissue. The source of the sample could be any mammal, such as, but not limited to, a human, primate, monkey, cat, dog, mouse, pig, cow, horse, sheep, and bat. Samples may also be obtained from any other source, such as air, water, soil, surfaces, food, beverages, nutraceuticals, clinical sites or products, industrial sites (including food processing sites) and products, plants and grains, cosmetics, personal care products, pharmaceuticals, medical devices, agricultural equipment and sites, and commercial samples.

In some embodiments, the target nucleic acids of interest are from one to many infectious agents (e.g., a bacteria, protozoan, insect, worm, virus, or fungus) that affect mammals, including humans. As a non-limiting example, the target nucleic acid of interests could be one or more nucleic acid molecules from bacteria, such as Bordetella parapertussis, Bordetella pertussis, Chlamydia pneumoniae, Legionella pneumophila, Mycoplasma pneumoniae, Acinetobacter calcoaceticus-baumannii complex, Bacteroides fragilis, Enterobacter cloacae complex, Escherichia coli, Klebsiella aerogenes, Klebsiella oxytoca, Klebsiella pneumoniae group, Moraxella catarrhalis, Proteus spp., Salmonella enterica, Serratia marcescens, Haemophilus influenzae, Neisseria meningitidis, Pseudomonas aeruginosa, Stenotrophomonas maltophilia, Enterococcus faecalis, Enterococcus faecium, Listeria monocytogenes, Staphylococcus aureus, Staphylococcus epidermidis, Staphylococcus lugdunensis, Streptococcus agalactiae, Streptococcus pneumoniae, Streptococcus pyogenes, Chlamydia tracomatis, Neisseria gonorrhoeae, Syphilis (Treponema pallidum), Ureaplasma urealyticum, Mycoplasma genitalium, and/or Gardnerella vaginalis. Also, as a non-limiting example, the target nucleic acid of interest could be one or more nucleic acid molecules from a virus, such as adenovirus, coronavirus HKU1, coronavirus NL63, coronavirus 229E, coronavirus OC43, severe acute respiratory syndrome coronavirus 2 (SARS-COV-2), human metapneumovirus, human rhinovirus, enterovirus, influenza A, influenza A/H1, influenza A/H3, influenza A/H1-2009, influenza B, parainfluenza virus 1, parainfluenza virus 2, parainfluenza virus 3, parainfluenza virus 4, respiratory syncytial virus, herpes simplex virus 1, herpes simplex virus 2, human immunodeficiency virus (HIV), human papillomavirus, hepatitis A virus (HAV), hepatitis B virus (HBV), hepatitis C virus (HCV), and/or human parvovirus B19 (B19V). Also, as a non-limiting example, the target nucleic acid of interest could be one or more nucleic acid molecules from a fungus, such as Candida albicans, Candida auris, Candida glabrata, Candida krusei, Candida parapsilosis, Candida tropicalis, Cryptococcus neoformans, and/or Cryptococcus gattii. As another non-limiting example, the target nucleic acid of interest could be one or more nucleic acid molecules from a protozoan, such as Trichomonas vaginalis.

In some embodiments, nucleic acids may be for non-infectious conditions, e.g., to be used for genotyping, including non-invasive prenatal diagnosis of, e.g., trisomies, other chromosomal abnormalities, and known genetic diseases such as Tay Sachs disease and sickle cell anemia. Other target nucleic acids of interest and samples include human biomarkers for cancer. Target nucleic acids of interest may include engineered biologics, including cells such as CAR-T cells, or target nucleic acids of interest from very small or rare samples, where only small volumes are available for testing. Still, other target nucleic acids and samples include forensic SNPs and STR markers from blood, saliva, semen, and oral swabs.

FIG. 1B is a perspective view of one embodiment of an automated sample prep device 100 as described herein; FIG. 1C is a perspective partially transparent view of the embodiment of the automated sample prep device 100 shown in FIG. 1B; and FIG. 1D is a front cross-sectional view of the embodiment of the automated sample prep device 100 shown in FIGS. 1B and 1C.

In any of FIGS. 1B-1D, sample prep device 100 comprises various components including sample chamber 110 having a cap or cover 112 and sample lysis region 116; support/housings 114, 122 and 126; a first diametrically poled magnet 120a; a second diametrically poled magnet 120b; fasteners 124; housing for electrical connections 130a and 130b to DC motors (not seen) contained in housings 134a and 134b; and screws 128a and 128b to position the motor (only 128b is seen in FIG. 1B). Additionally, in FIG. 1D is seen a support and positioning element 144 for sample chamber 110.

FIG. 1E is a perspective partially transparent view of a second embodiment of the automated sample prep device 160 described herein; and FIG. 1F is a cross-sectional perspective view of the second embodiment of the automated sample prep device 160 shown in FIG. 1E. Much like the embodiment of the automated sample prep device 100, automated sample prep device 160 comprises various components including sample chamber 110 having a cap or cover 112 and sample lysis region 116; support/housings 114, 122, 126, and 136; a first diametrically poled magnet 120a; a second diametrically poled magnet 120b; fastener 142; fasteners 124 and 138; and a support and positioning element 144 for sample chamber 110. However, the embodiment of the automated sample prep device 160 shown in FIGS. 1E and 1F comprises stepper motors 154a and 154b instead of the DC motors used in the embodiments shown in FIGS. 1B-1D. Thus, automated sample prep device 160 comprises housing for encoders 150a and 150b for stepper motors 154a and 154b. In FIG. 1F, actuators 156a and 156b for the stepper motors are seen.

FIG. 1G is a front perspective view of a third embodiment of a sample prep device 170, with a single motor housing 134 for a single motor (not shown) further comprising a first diametrically poled magnet 120a; a second diametrically poled magnet 120b; a support and positioning element 144 for sample chamber 110; and three heat conducting elements 158 surrounding sample chamber 110 on three sides of sample chamber 110. Motor housing(s) (not seen) contain drive motor(s) that generate rotational motion of the first and second diametrically poled magnets 120a and 120b. The motor(s) (not seen) typically includes a drive shaft that engages a lower end (or, in some embodiments, an upper end) of the diametrically poled magnets.

FIG. 1H is a front perspective view of a fourth embodiment of a sample prep device 180, comprising a single drive motor 164; a first diametrically poled magnet 120a; a second diametrically poled magnet 120b; a sample chamber 110 having a cap or cover 112; housing 126; toggle clamp 162; and Hall effect sensor 166. Hall effect sensor 166 is an integrated circuit that transduces the magnetic field to an electrical signal to allow for sensing, positioning and speed detection of at least one of the diametrically poled magnets 120a and 120b. FIG. 1I is a front perspective view of a “commercial” fifth embodiment of a sample prep device 190 comprising a first diametrically poled magnet 120a; a second diametrically poled magnet 120b; a sample chamber 110 having a cap or cover 112; housing 126; toggle clamp 162; and keypad 168.

In FIGS. 1B-1F there are two motors present, one for each diametrically poled magnet which allows for rotation of both magnets. FIGS. 1G and 1H, on the other hand, comprises a single motor and only one of the diametrically opposed magnets rotates; that is, one of the diametrically poled magnets rotates and the other is stationary. In some embodiments, the magnet that is held stationary may be “tuned” by a small rotation of its field to locate the best interaction location.

The motor, in some embodiments, is a brushless DC type drive motor with built-in drive controls that can be configured to maintain a constant revolution per minute (RPM) between 0 and about 8000 RPM, or between 500 and about 6000 RPM, or between 1000 and about 4000 RPM. Alternatively, other motor types such as stepper (as shown in FIGS. 1E and 1F), servo, or brushed DC motors can be used. A stepper motor design uses an axially magnetized permanent magnet that is sandwiched between two teethed rotor cups. By magnetizing the permanent magnet, the teeth of the two rotor cups become magnetic poles of opposite polarity. A stepper motor can be commanded to move to a position, stop, then hold the position for a period of time before moving on to a second position and so on. An alternative to a stepper motor is a servo motor, which uses a radially magnetized rotor instead of teeth and requires the use of encoder feedback. Where a stepper motor can be commanded to move to a position, stop, then hold, a servo motor needs to “hunt” for a position with encoder feedback. Brushed DC motors have coils in their center rotating around permanent magnets while brushless DC motors have a permanent magnet in their center that rotates around the coils.

The motor(s) are configured to deliver at least 20 watts of power, or at least 10 watts of power, or at least 2 watts of power.

Optionally, one or both of the motors may also have direction control to allow reversing of the rotational direction and a tachometer to sense and report actual RPM. In other examples, the motor may generate oscillating motion by reversing the direction of rotation at a predetermined frequency. The motor typically is communicatively coupled through a wired or wireless communication network to a processing system configured to control the operation of the motor, which can include executing protocols programmed into the processor and/or provided by user input. Motors are available from a number of different suppliers, including ElectroCraft, Inc. (Stratham NH, USA), Bodine Electric Company (Northfield IL, USA), Dumore Corp. (Mauston WI, USA), Oriental Motor USA (Torrence CA, USA), and Industrial Parts and Electric Motors (Memphis TN, USA).

In many embodiments, supports or housings 114, 122, 126 and 136 (support/housing 136 is seen only in FIGS. 1E and 1F) are fabricated from a robust material including aluminum, stainless steel, and/or other material, including plastics. These supports or housings—or portions thereof—can be created through various techniques including, e.g., metal fabrication, injection molding, and creation of structural layers that are fused.

The sample chamber may be fabricated from bio-compatible glass, plastics or other materials having no or low magnetic permeability and that are robust enough to survive collisions from the spheres. In many embodiments, the sample size is very small and, e.g., small Eppendorf or other types of sample tubes are used for the sample chamber, including those with conical bottoms or with flat bottoms. In some embodiments the sample chamber may be reusable; however, in most embodiments, the sample chamber 110 is consumable. The diameter of the sample lysis region 116 of the sample chamber 110 is the point where the number of spheres used should be determined (described in detail below). The cap or cover 112 shown in FIGS. 1B-1E is optional; however, employing a cap or cover reduces the risk of contamination and loss of sample. Further, the sample chamber can be oriented either horizontally or vertically, allowing for flexibility in instrument compatibility.

Aside from the sample chamber, the other components of the sample prep device (i.e., supports and housings), in most aspects, are reusable and the automated sample prep device can function as a stand-alone device or as a module in an automated sample processing and nucleic acid detection system.

The diametrically poled magnets are most often configured as cylindrical or ring magnets but can comprise any magnet that can be rotated. The magnets are magnetized perpendicular to the axis of the center hole and are often fabricated from neodymium. The magnets used in a typical automated sample prep device are configured to impart between a 2-pound magnetic pull force and a 10-pound pull force, depending on the sample size, sample chamber size and diameter and number and size of metal spheres employed (described in detail below). Ring magnets are available widely from a number of sources including ATech (Beijing, PRC); Dexter Magnetic Technologies (Elk Grove Village IL, USA), K&J Magnetics (Pipersville PA, USA), Industrial Magnetics, Inc. (Boyne City MI, USA), and MagnetShop.com (Culver City CA, USA).

In some embodiments—as shown above in FIG. 1G at 158—the sample chamber 110 is proximate to one or more heat transferring elements so as to rapidly heat and cool sample chamber 110, which is important for certain embodiments of virus lysis in a lysis only/no purification workflow. The heat transferring elements 158 may be fabricated from copper and thermal ramping and target temperature may be controlled through monitoring a thermal sensor such as a thermistor (not shown) included proximal to sample chamber 110. In a particular embodiment, the heater such as a Peltier device is designed heat (or cool) the contents of sample chamber 110 to a temperature of between 25° and 95° C.

In operation, the sample chamber of the automated sample prep device is “loaded” with a sample putatively comprising cells or viral particles of interest as well as lysis/binding beads that comprise 10% to 60% of the total sample volume. In addition, “X” number of spheres are added such that the spheres will exceed the span of the diameter of the sample chamber (i.e., in sample lysis region 116 of the sample chamber. For example, for a typical 2 mL Eppendorf tube, three 2.7 mm spheres span the diameter of the tube and one additional 2.7 mm sphere is added to create the chaotic interaction between the spheres. If the magnets are held steady, the spheres line up in an orderly fashion in the magnetic field, north to south. However, as one or both of the diametrically poled magnets are rotated, the magnetic field changes causing chaotic movement of the spheres, and, in turn, chaotic movement of the lysis/binding beads in the sample. As the lysis/binding beads are shuffled and tumbled by the movement of the spheres, cells and/or viral particles present in the sample are lysed thereby releasing nucleic acids.

As the bacteria, viruses, fungi or other cells/organisms are lysed, the nucleic acids from these source organisms are fragmented into lengths of nucleic acids approximately 30 bp to 12,000 bp depending on the size of the cellular or viral genome, with the level of fragmentation dependent upon extent of chaotic motion induced by the size and type of spheres and lysis/binding beads, lysis/binding bead volume to sample volume, and temperature. The temperature maybe increased to 95° C. either during lysis, subsequent to lysis or during elution of the nucleic acids during purification. It has been demonstrated that bead beating E. coli cells for 1-3 minutes resulted in fragment sizes that look to be 500 bp-10 kb (see Proctor, et al., researchgate.net/publication/334045102, June 2019). It has also been demonstrated that heating E. coli DNA at 95° C. for 5 minutes results in fragment sizes of 500 bp-1000 bp (see Yang and Hang, J. of Biomolecular Techniques, 24:98-103 (2013)). Human DNA fragments appear as a smear from 650 bp-12,000 bp when heated at 95° C. for 2 minutes, while 18 minutes of heating resulted in a smear ranging from 100 bp-500 bp. Thus, heating may be employed along with the bead beating action of the sample prep device.

Bead beating is a known effective method of cell lysis which is used to disrupt virtually any biological sample by agitating the sample (and thus the source organisms/cells) with a lysing matrix (i.e., a grinding medium or beads) in a bead beater instrument, here, the automated sample prep device. The lysing matrix refers to the physical beads used to lyse and homogenize the samples and the lysing matrix chosen depends on the size and physical properties of the source organisms (e.g., whether there is a cell wall, if the source organism is a spore). Bead shape determines how cells are disrupted and influences how aggressive the lysing process is. In addition to cell lysis, bead beating can shear nucleic acids into fragments to improve sensitivity for detection.

Lysis beads are classified as spherical, utilizing impaction as the leading force, and angular, generating mechanical shear forces to chop and cut samples. The size of bead used is based on the properties of the material being disrupted and the compatibility with the device hardware, fluidics and processing steps. Typically, 0.1 mm diameter beads are used to lyse bacteria and viruses. 0.5 mm diameter beads are used to lyse yeast and 1-2.5 mm diameter beads are used for plant or animal tissue.

Finally, beads are fashioned from many different materials, which vary depending on desired hardness, durability and chemical resistance including but not limited to glass, zirconium silicate, zirconium oxide, stainless steel, silica, various ceramics, and silicon carbide. For example, bacteria, and algae comprise a soft cell wall and small cell size and lysing matrices that are spherical and small made of silica, ceramics or glass are appropriate; whereas yeast, fungi, and environmental samples such as soil and wood have harder cells walls and larger cell size and lysing matrices that deliver medium sheer and high impact such as silicon carbide or zirconium oxide are appropriate. In the present methods, the bead type must not only induce lysis and nucleic acid fragmentation, but in some embodiments, bind nucleic acids for purification.

The spheres utilized again are of a size that several spheres will exceed the span of the diameter of the sample chamber (i.e., in the sample lysis region 116 of the sample chamber). In one example, three 2.7 mm span almost span the diameter of a 2 mL Eppendorf tube; thus, at least four stainless steel 406 beads would be employed in the lysis procedure. The spheres preferably have a magnetic permeability where the spheres will react to the magnetic field created by the diametrically poled magnets but possess a relatively low magnetic permeability; for example, relative permeabilities of K~1.002 to 1.005 are typically reported for 304 stainless steel and 316 stainless steel in their annealed state, where a magnetic permeability of 1.000 is non-magnetic. Thus, spheres of any bio-inert material may be used in the automated sample prep device as long as the magnetic permeability exceeds 1.000.

FIG. 1J is an exemplary system diagram for the sample prep device. In this exemplary system diagram, there is a 9V power supply driving a DC buck stepdown switched-mode converter, which decreases voltage from the input power supply (9V to 5V) while increasing current to an ARDUINO™ R3 microcontroller board interface. The ARDUINO™ R3 microcontroller board interface interfaces with an ADAFRUIT™ touch-panel and display screen, as well as receives input from a Hall effect sensor, which, as described above, is an integrated circuit that transduces a magnetic field to an electrical signal to sense, position and control the speed of the single rotating magnet (in this embodiment). ARDUINO™ R3 microcontroller board interface also interfaces with a DC motor driver that controls the speed and directionality of the DC motor.

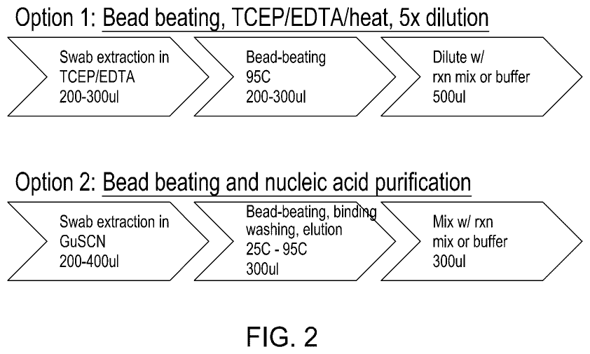

FIG. 2 shows two exemplary workflows for bead beating using the automated sample prep device. The first option employs bead beating only, where the second option employs bead beating, nucleic acid binding, washing and elution. In the first option, the first step, sample extraction, is performed in a sample buffer comprising Tris (2 carboxyethyl)phosphine (TCEP) and ethylenediaminetetraacetic acid (EDTA), where TCEP is used to inhibit RNase activity and EDTA is used to sequester divalent cations to inhibit DNase activity, resulting in a sample extract of 200-300 μL. Lysis beads are added to the sample extract to increase the volume of the sample extract by approximately 10 to 60 percent and an appropriate number of spheres are added as well.

The sample extract is then subjected to bead beating in the automated sample prep device at between 1000-7000 rpm for approximately 1-5 minutes at 95° C. to lyse cells or virus present in the sample, to fragment the nucleic acids released upon lysis and to provide an additional mechanism to inhibit nuclease activity. The high temperature allows for rapid RNase and DNase inactivation and virus lysis. Following bead beating, the sample extract is then diluted by increasing the volume of the sample extract to approximately 60 to 150 percent with, e.g., buffer or a desired reaction mix, thereby diluting the concentration of any inhibitors of downstream processes.

In the second option, the first step, sample extraction, is performed in a sample buffer comprising guanidinium thiocynate (GuSCN), which inhibits DNase and RNase activity and, as a chaotropic agent, promotes the binding of nucleic acids to the lysis/binding beads that are added in the next step. Other useful chaotropic agents include n-butanol, ethanol, guanidinium isothiocyanate, guanidinium chloride, lithium percholate, lithium acetate, magnesium chloride, phenol, sodium dodecyl sulfate, thiourea and urea. At this point, the volume of the sample extract will be approximately, 200-400 μL. In another embodiment, sample extraction is performed in a buffer of SDS and the extract is mixed with GuSCN. Lysis beads are added to the sample extract to increase the volume of the sample extract by approximately 10 to 60 percent and an appropriate number of spheres are added as well. The sample extract is then subjected to bead beating in the automated sample prep device at 1.5-4.0 volts for approximately 1-5 minutes. In this second option, the bead beating promotes cell lysis, nucleic acid fragmentation as well as nucleic acid binding to the lysis/binding beads.

After bead beating, the lysis/binding beads are washed at least once in buffer or in water—using agitation or flow-through only—and following washing, buffer is added to the nucleic acids and the nucleic acids are then eluted from the beads. Elution can be effected by, e.g., adding a buffer containing compounds that promote unbinding of the nucleic acids to the lysis/binding beads, raising the temperature of the extract, and/or agitating the lysis/binding beads by bead beating. Note that the workflows in FIG. 2 are exemplary only, where, e.g., different protein inhibitors, chaotropic agents, volumes and temperatures may be substituted for those shown.

### EXAMPLES

The following examples are put forth so as to provide those of ordinary skill in the art with a complete disclosure and description of how to make and use the present invention and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Preparation of Sample Nucleic Acids, Bead Beating Only

The workflows described herein contemplate collecting samples via nasal or nasopharyngeal swabs and detecting viruses and/or gram negative and/or gram positive bacteria source organisms. Further, the workflows work with small sample volumes in the automated sample prep device resulting in a nucleic acid sample in less than 10 minutes.

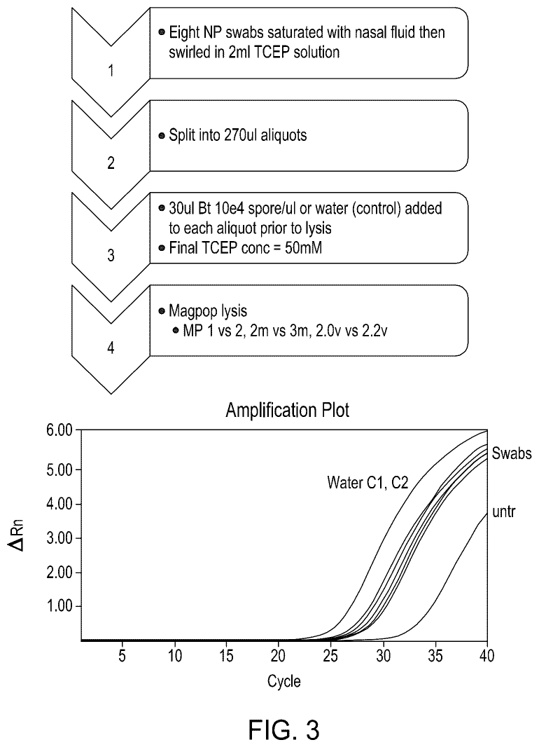

FIG. 3 depicts a first exemplary workflow for sample preparation as well as the results of an experiment performed to evaluate effectiveness of nucleic acid recovery. Note that the volumes recited in this Example 1 are exemplary and may be doubled, halved, or increased or decreased by X % depending on the type of swab used for collection and the source of the sample. To approximate actual sample collection and samples, eight nasopharyngeal swabs were saturated with nasal fluid (available from Biochemazone (Alberta, Canada); Inveniolife (Mumbai, India); and TMALAB (Seoul, Korea)) and swirled in 2 mL (pooled) buffer solution comprising 55 mM TCEP. The resulting sample solution was split into seven 270 μL aliquots. An additional 270 μL aliquot comprising only water was used as a control. In a next step, 30 μL of Bacillus thuringiensis (“Bt”) spores at a concentration of 10⁴ spores/μL was added to each aliquot prior to lysis. 600 mg 0.1 mm zirconia glass beads (BioSpec, Bartelsville, OK USA) and four 2.7 mm 306 stainless steel beads (McMaster Carr, Robbinsville NJ, USA) were added to each sample aliquot and each aliquot was subjected to a bead beating procedure in one of two automated sample prep devices (M1 or M2) at 2.0 volts or 2.2 volts for 2 minutes or 3 minutes.

Following dilution, qPCR was performed to quantify the amount of DNA recovered and the results are shown in FIG. 3. Use of automated sample prep device 2 (M2) at 2.0 volts for 2 minutes produced the best result (data not shown). Note also in the amplification plot, the results for the swab samples resulted in tight replicates.

### Example II: Preparation of Sample Nucleic Acids, including Purification

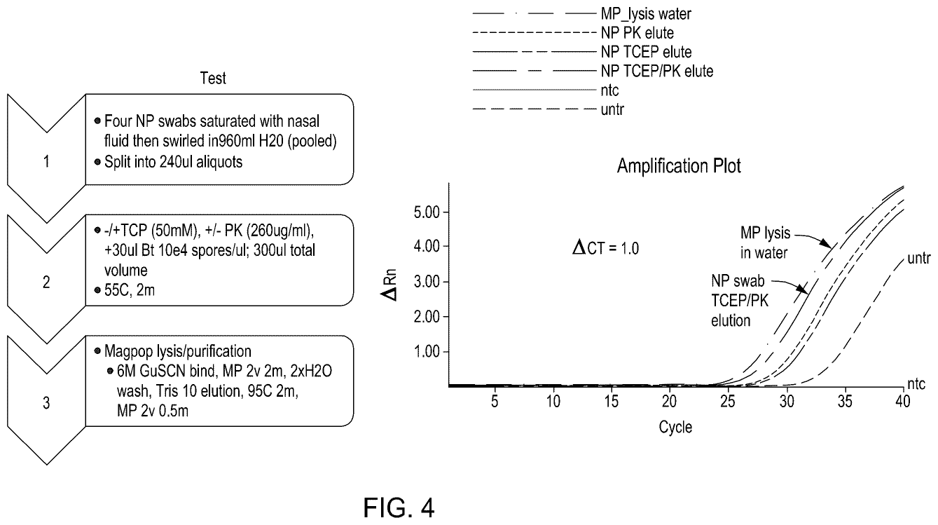

FIG. 4 is a graphic depiction of a second exemplary workflow for sample preparation, with this workflow comprising the use of TCEP (Tris(2-carboxyethyl) phosphine hydrochloride) and Proteinase K (PK) to inactivate DNases and RNases; guanidinium thiocynate (GuSCN) to also inactivate nucleases and to facilitate binding of nucleic acids to the lysis/binding beads; water washes to clear non-nucleic acid lysate material, Proteinase K (PK), TCEP, and guanidinium thiocyanate (GuSCN) from the lysis/binding beads; and heating to 95° C. to increase the elution efficiency of nucleic acids from the lysis/binding beads following purification. In a first step, four nasopharyngeal swabs were saturated with nasal fluid (available from Biochemazone (Alberta, Canada); Inveniolife (Mumbai, India); and TMALAB (Seoul, Korea)), swirled in 960 μL (pooled) buffer solution, and split into four 240 μL aliquots. TCEP was added to a final concentration of 50 mM in two aliquots and Proteinase K (PK) was added to a final concentration of 260 μg/mL in two aliquots for a total volume of 300 μL per aliquot. The aliquots thus comprised 1) Bt spores in water; 2) +TCEP/−PK; 3) −TCEP/+PK; and 4) +TCEP/+PK.

In a next step, 30 μL of Bacillus thuringiensis (“Bt”) spores at a concentration of 10⁴ spores/μL was added to each aliquot prior to lysis. All four aliquots were heated to 55° C. for two minutes. Following incubation, 600 mg 0.1 mm zirconia glass beads (BioSpec, Bartelsville, OK USA), four 2.7 mm 306 stainless steel beads (McMaster Carr, Robbinsville NJ, USA), and GuSCN to a final concentration of 3M were added to each aliquot with the exception of the water control. The water control is a lysis only control with no purification. The elution PCR Ct is compared to the lysis only PCR Ct to determine % recovery. The aliquots were subjected to a bead beating procedure in the automated sample prep device described above at 2.0 volts for 2 minutes, followed by two H₂O washes. After the washes, the lysis/binding beads were resuspended in buffer to a final concentration of 10 mM Tris pH10, heated to 95° C. for 2 minutes, and subjected to bead beating at 2.0 volts for 30 seconds to facilitate elution of the nucleic acids from the lysis/binding beads.

Following elution, qPCR was performed to quantify the amount of DNA recovered. Note that the amplification plot in FIG. 4 indicates that the aliquot with both TCEP and PK resulted in 50% DNA recovery. The ΔCT for the aliquots comprising only TCEP or only PK was much higher than that for the aliquot comprising both TCEP and PK, as the TCEP and PK help break down the mucus in the sample rendering the bead-beating more effective since the swabs were extracted in volumes 3-10 times lower than used by conventional protocols. This enables improved limit of detection, since in conventional approaches swabs are extracted in volumes ranging from 1-3 mL and typically only a fraction of this volume is processed for microbial nucleic acid detection, thereby decreasing sensitivity.

### Example III: Preparation of Sample Nucleic Acids, DNA and RNA

In a third protocol, extraction of both DNA and RNA was tested. The following were combined for each nasopharyngeal swab sample: 300 μL 3M guanidinium thiocyanate (GuSCN) in 50% ethanol; 30 μL MS2 bacteriophage at a concentration of 10² pfu/μL; 2 μL (1 μg/μL) carrier RNA; and 300 μL Bt spores at a concentration of 10e3 spores/μL. 600 mg 0.1 mm zirconia glass beads (BioSpec, Bartelsville, OK USA) and four 2.7 mm 306 stainless steel beads (McMaster Carr, Robbinsville NJ, USA) were added to each aliquot, and bead beating in the automated sample prep device described above was performed at 1.5 volts for 2 minutes, followed by two H₂O washes. After the washes, the lysis/binding beads were resuspended in buffer to a final concentration of 10 mM Tris pH10, heated to 95° C. for 2 minutes, and subjected to a final bead beating procedure at 2.0 volts for 30 seconds to facilitate elution of the nucleic acids from the lysis/binding beads. During the initial bead beating procedure, the orientation of the magnets was varied at 6 o'clock (standard), at 9 o'clock and at 12 o'clock, to determine if the non-spinning stationary magnet orientation was critical to achieve spore lysis or better binding since the level of agitation varies with the non-spinning magnet orientation.

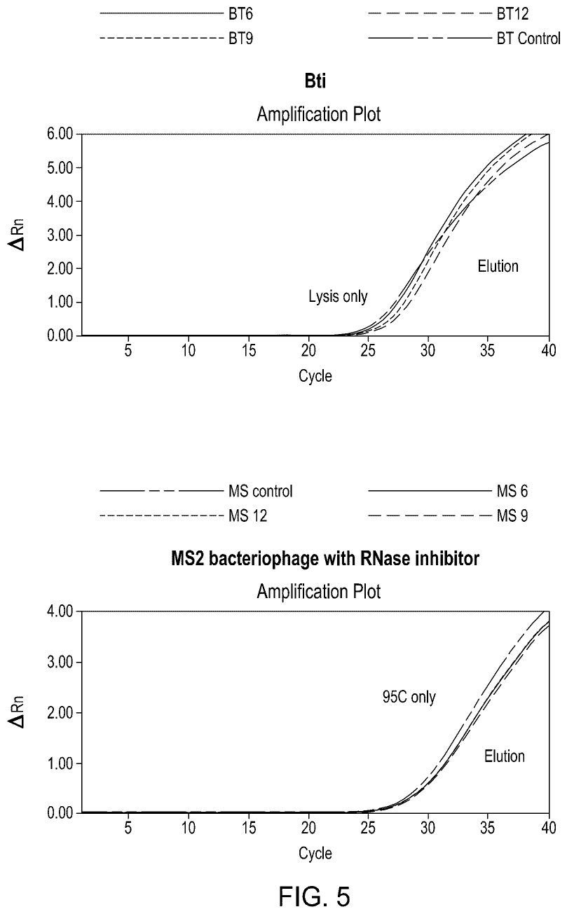

Following elution, qPCR was performed to quantify the amount of DNA and RNA recovered. FIG. 5 comprises two amplification plots showing the recovery of DNA (Bti) and RNA (MS2 bacteriophage with murine RNase inhibitor (New England Biolabs®, Ipswich MA USA)). For Bti, the fixed magnet polarity orientation was best at position 6 (Bt6), good at position 9 (Bt9), and subpar at position 12 (Bt12). For MS2 bacteriophage, orientation was good at position 6 (MS6), and very good at both position 9 (MS9) and position 12 (MS12). Positions 6, 9, and 12 each represent a 45 degree rotation in the fixed magnet orientation.

### Example IV: Fragmentation of Nucleic Acids

Bacteriophage lambda DNA (48,503 bp) was used to estimate the extent of DNA fragmentation by the automated sample prep device alone, heat treatment alone, and a combination of the automated sample prep device and heat treatment. The results of the time course of DNA fragmentation using the automated sample prep device alone are seen at left, where good fragmentation is seen at 6 minutes. At center is the time course of DNA fragmentation using heat treatment (~95° C.) in the absence of salt in the solution, where again, good fragmentation is seen at 6 minutes. Heat treatment denatures double-strand DNA to single-strand DNA, resulting in a migration shift from 0 minutes to 1 minute (arrow). Finally, at right are the results of replicates of combined treatment using the automated sample prep device and heat, for a total treatment time of 2 minutes, 50 seconds, with a temperature of approximately 87° C. achieved at 1 minute, 20 seconds. The results for the combined treatment achieved at 2 minutes 50 seconds are comparable to 6 minutes of treatment with the automated sample prep device or with heating at 95° C. alone.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

We claim:

1. A device for automated preparation of nucleic acids from a sample comprising:
a first rotatable diametrically poled magnet;
a second rotatable diametrically poled magnet, wherein the second magnet is set apart from the first rotatable magnet, and wherein one or both of the first and second magnets rotates around a vertical axis;
at least one motor configured to rotate at least one of the first and second magnets; and
a sample chamber positioned between the first and second rotatable magnets, wherein the sample chamber comprises lysis beads and X spheres, wherein the spheres have a magnetic permeability greater than 1.000, and wherein a diameter of the X spheres exceeds a diameter of the sample chamber.

2. The device of claim 1, wherein the first and second rotatable magnets are cylindrical with a central hole vertically traversing the first and second rotatable magnets.

3. The device of claim 2, wherein the first and second rotatable magnets are fabricated from neodymium.

4. The device of claim 1, wherein the sample chamber has a volume of 0.5 mL to 10.0 mL.

5. The device of claim 4, wherein the sample chamber has a volume of 1.0 mL and 5.0 mL.

6. The device of claim 5, wherein the sample chamber has a volume of 1.5 mL and 2.5 mL.

7. The device of claim 1, wherein the first and second magnets are configured to impart between a 1.5-pound magnetic pull force and a 10.0-pound pull force.

8. The device of claim 7, wherein the first and second magnets are configured to impart between a 2.0-pound magnetic pull force and a 7.0-pound magnetic pull force.

9. The device of claim 1, wherein the spheres are metal spheres.

10. The device of claim 9, wherein the spheres are stainless steel.

11. A method for isolating nucleic acids from cells or viral particles in a sample comprising the steps of:
obtaining a sample;
obtaining the sample prep device of claim 1;
transferring the sample to the sample chamber of the sample prep device;
adding lysing buffer to the sample in the sample chamber;
rotating at least one of the rotatable magnets at 500-6000 RPM for 0.5-5 minutes;
diluting the sample with buffer; and
collecting the sample.

12. The method of claim 11, wherein the lysing buffer comprises EDTA, TCEP or DTT.

13. The method of claim 11, wherein the rotating step is performed at 95° C.

14. The method of claim 11, wherein a volume of the sample is 200-300 μL

15. The method of claim 11, wherein the lysis beads comprise 20-50% of the sample volume after the transferring step.

16. The method of claim 11, wherein the lysing buffer comprises a chaotropic agent.

17. The method of claim 16, wherein the chaotropic agent is guanidine isothiocyanate.

18. The method of claim 16, wherein after the adding step and before the rotating step, the method further comprises a step of heating the sample.

19. The method of claim 16, wherein after the rotating step and before the diluting step, the method further comprises a step of washing the lysis beads.

20. The method of claim 17, wherein after the washing step and before the diluting step, the method further comprises a step of suspending the lysis beads in suspension buffer and heating the suspension buffer to 95° C.

21. The method of claim 20, wherein the suspension buffer comprises Tris at pH 10.

22. The method of claim 20, wherein after the suspending step and before the diluting step, the method further comprises a step of rotating at least one of the rotatable magnets 500-6000 RPM for 0.5-5 minutes for 0.5-5 minutes.
